(19) United States
(12) Patent Application Publication
(10) Pub. No.: US 2026/0358353 A1
(43) Pub. Date: Nov. 12, 2026

(54) PROTEIN MARKER FOR ASSESSING AND TREATING NEURODEGENERATIVE DISEASES

(71) Applicants: The Hong Kong University of Science and Technology, Kowloon, Hong Kong (CN); HONG KONG CENTER FOR NEURODEGENERATIVE DISEASES LIMITED, Shatin, Hong Kong (CN)

(72) Inventors: Nancy Yuk-Yu IP, Clear Water Bay, Kowloon, Hong Kong SAR (CN); Kit Yu FU, Clear Water Bay, Kowloon, Hong Kong SAR (CN); Yuanbing JIANG, Clear Water Bay, Kowloon, Hong Kong SAR (CN); Hye-bin UHM, Clear Water Bay, Kowloon, Hong Kong SAR (CN)

(21) Appl. No.: 19/118,341

(22) PCT Filed: Oct. 7, 2023
(86) PCT No.: PCT/CN2023/123209

§ 371 Date: Apr. 3, 2025

Related U.S. Application Data
(60) Provisional application No. 63/413,969, filed on Oct. 7, 2022.

(51) Int. Cl. G01N 33/68

(52) U.S. Cl. CPC G01N 33/6896; G01N 2800/2821; G01N 2800/2835; G01N 2800/50

(57) ABSTRACT

Provided is a protein marker Nell-1, which is present in a person's blood sample in an amount that is correlated with neurodegenerative disorders such as Alzheimer's Disease (AD), Mild Cognitive Impairment (MCI), and Parkinson's Disease (PD). Corresponding diagnostic and treatment methods for these neurodegenerative disorders as well as kits for diagnosing or treating the neurodegenerative disorders are also provided.

### RELATED APPLICATIONS

This application claims priority to U.S. Provisional Patent Application No. 63/413,969, filed Oct. 7, 2022, the contents of which are hereby incorporated by reference in the entirety for all purposes.

### BACKGROUND OF THE INVENTION

Brain diseases such as neurodegenerative diseases and neuroinflammatory disorders are devastating conditions that affect a large subset of the population. Many are incurable, highly debilitating, and often result in progressive deterioration of brain structure and function over time. Disease prevalence is also increasing rapidly due to growing aging populations worldwide, since the elderly are at high risk for developing these conditions. Currently, many neurodegenerative diseases and neuroinflammatory disorders are difficult to diagnose due to limited understanding of the pathophysiology of these diseases. Meanwhile, current treatments are ineffective and do not meet market demand; demand that is significantly increasing each year due to aging populations. For example, Alzheimer's disease (AD) is marked by gradual but progressive decline in learning and memory, and a leading cause of mortality in the elderly. Increasing prevalence of AD is driving the need and demand for better diagnostics. According to Alzheimer's Disease International, the disease currently affects nearly 40 million people globally, but the number of cases is projected to triple in the coming three decades. One of the countries with the fastest elderly population growth is China. Based on population projections, by 2030 one in four individuals will be over the age of 60, which will place a vast proportion at risk of developing AD. In fact, the number of AD cases in China doubled from 3.7 million to 9.2 million from 1990-2010, and the country is projected to have 22.5 million cases by 2050. Hong Kong's population is also aging quickly. It is estimated that the elderly aged 65+ will make up 24% of the population by 2025, and 39.3% of the population by 2050. The number of AD cases is projected to rise to more than 330,000 by 2039.

More worrisome is that, despite the increase in AD prevalence, many people fail to receive a correct AD diagnosis. According to Alzheimer's Disease International's World Alzheimer' Report 2015, in high-income countries only 20-50% of dementia cases are documented in primary care. The rest remain undiagnosed or incorrectly diagnosed. This ‘treatment gap’ is much more significant in low- and middle-income countries. Without a formal diagnosis, patients do not receive the treatment and care they need, nor do they or their care-givers qualify for critical support programs. Early diagnosis and early intervention are two important means of narrowing the treatment gap. Thus, early diagnostic tools that can determine disease risk both quickly and accurately have significant therapeutic value on many levels. Research has confirmed that AD affects the brain long before actual symptoms of memory loss or cognitive decline actually manifest. To this date, however, there are few diagnostic tools for early detection, see, e.g., WO2021/228125. In most cases, by the time a patient is diagnosed with AD using methods currently in practice, which involves subjective clinical assessment, the pathological symptoms are typically already at an advanced state. As such, for the purpose of improving AD treatment and long-term management, there exists an urgent need for developing more new and effective methods for early diagnosis of AD in a patient or for detecting an increased risk of a patient developing AD at a future time. This invention addresses this and other related needs by disclosing novel methods and kits related to the use of circulating protein Nell-1 as a biomarker for assessing individual risk of developing neurodegenerative disorders such as the Alzheimer's Disease (AD), Mild Cognitive Impairment (MCI), and Parkinson's Disease (PD) and as a therapeutic target for treating neurodegenerative disorders.

### BRIEF SUMMARY OF THE INVENTION

The invention relates to the discovery of novel plasma protein markers associated with a neurodegenerative disorder such as the Alzheimer's Disease (AD), Mild Cognitive Impairment (MCI), and Parkinson's Disease (PD). The invention thus provides methods and compositions useful for diagnosis of these neurodegenerative disorders as well as for indicating therapeutic efficacy of an agent for treating the neurodegenerative disorders. As such, in a first aspect, the present invention provides a method for assessing a subject's risk of developing a neurodegenerative disorder such as AD, MCI, or PD at a future time. The method includes the following steps: first, comparing the subject's plasma or serum or whole blood level of Nell-1 protein with a standard control level of Nell-1 protein found in the plasma or serum or whole blood of an average healthy subject not suffering from or at increased risk for the neurodegenerative disorder. Second, either detecting a decrease in the subject' plasma or serum or whole blood level of Nell-1 protein from the standard control level, and determining the subject as suffering from or having an increased risk for the neurodegenerative disorder; or, as an the alternative to the second step, a third step of detecting no decrease in the subject' plasma or serum or whole blood level of Nell-1 protein from the standard control level, and determining the subject as not suffering from or having no increased risk for the neurodegenerative disorder. In some embodiments, this method further comprises, prior to the first step, a step of measuring the plasma or serum or whole blood level of Nell-1 protein. In some embodiments, the method further comprises, prior to the above-mentioned measuring step, a step of obtaining a plasma or serum or whole blood sample from the subject. In some embodiments, the method further comprises, after the third step, a step of comparing the subject's plasma or serum or whole blood level of Nell-1 protein as measured at a later time (i.e., at a second time point) with the subject's plasma or serum or whole blood level of Nell-1 protein in the first step (i.e., at the original time point, or an earlier or first time point), wherein a higher plasma or serum or whole blood level of Nell-1 protein as measured at the later time indicates improvement of the neurodegenerative disorder, and wherein a lower plasma or serum or whole blood level of Nell-1 protein as measured at the later time indicates deterioration of the neurodegenerative disorder. In some embodiments, between the third step (or the first time) and the later time (or the second time), the subject has been administered a therapeutic agent intended for treating the neurodegenerative disorder. In some embodiments, the neurodegenerative disorder for which risk is being assessed is Alzheimer's Disease (AD). In some embodiments, the neurodegenerative disorder being assessed is Mild Cognitive Impairment (MCI) or Parkinson's Disease (PD).

In a second aspect, the present invention provides a method for assessing the level of comparative risk for a neurodegenerative disorder such as AD, MCI, or PD among two or more subjects. The method includes these steps: first, comparing the first subject's plasma or serum or whole blood level of Nell-1 protein with the second subject's plasma or serum or whole blood level of Nell-1 protein; second, detecting the second subject's plasma or serum or whole blood level of Nell-1 protein lower than the first subject's plasma or serum or whole blood level of the Nell-1 protein; and third, determining the second subject as having lower severity or lower risk of the neurodegenerative disorder than the first subject. In some embodiments, the neurodegenerative disorder for which the risk is assessed is Alzheimer's Disease (AD). In some embodiments, the neurodegenerative disorder for which the risk is assessed is Mild Cognitive Impairment (MCI) or Parkinson's Disease (PD). In some embodiments, the claimed method further comprises, prior to the first step, measuring the plasma or serum or whole blood level of Nell-1 protein. In some embodiments, the method as mentioned above, further comprises, prior to the measuring step, a step of obtaining a plasma or serum or whole blood sample from the subject.

In a third aspect, the present invention provides a kit for assessing risk for neurodegenerative disorders such as AD, MCI, and PD in a subject or for assessing therapeutic efficacy of a treatment regimen for these neurodegenerative disorders. The kit includes a first container containing a first reagent capable of determining the subject's plasma or serum or whole blood level of Nell-1 protein, and, optionally, a second container containing a second reagent capable of determining the subject's plasma or serum or whole blood level of phosphorylated-Tau-181 (pTau181) or neurofilament light polypeptide (NfL). In some embodiments, the kit includes reagents capable of determining the subject's plasma or serum or whole blood level of two proteins pTau181 and NfL. In some embodiments, the kit further includes a standard control reflecting the level of Nell-1 protein found in the plasma or serum or whole blood of an average healthy subject not suffering from and not having an increased risk for the neurodegenerative disorder. In some embodiments, the kit described above or herein is intended for assessing the risk of or the therapeutic efficacy of a treatment regimen for the neurodegenerative disorder of Alzheimer's Disease (AD), Mild Cognitive Impairment (MCI), or Parkinson's Disease (PD).

In a fourth aspect, the present invention provides a method for treating a neurodegenerative disorder or for reducing the risk for such a disorder. The method includes the step of administering to a subject in need thereof an effective amount of (1) a Nell-1 enhancing agent that enhances Nell-1 protein expression or activity; or (2) a Nell-1 receptor enhancing agent that enhances Nell-1 receptor expression or activity. In some embodiments, the Nell-1 enhancing agent is Nell-1 protein or a nucleic acid encoding the Nell-1 protein. In some embodiments, the Nell-1 enhancing agent is a Nell-1 receptor, a nucleic acid encoding the Nell-1 receptor, or an agonist of the Nell-1 receptor. In some embodiments, the step of administration comprises brain-targeting delivery of the Nell-1 enhancing agent or the Nell-1 receptor enhancing agent. In some embodiments, where a nucleic acid encoding Nell-1 protein or the Nell-1 receptor is administered, the nucleic acid is formulated in a lipid nanoparticle composition for delivery. In some embodiments, the nucleic acid encoding Nell-1 protein or the Nell-1 receptor is introduced by gene editing. In some embodiments, this treatment method as claimed further comprises, before and/or after the administering step, measuring the subject's plasma or serum or whole blood level of Nell-1 protein. In some embodiments, the claimed method further comprises, prior to the measuring step or steps, obtaining a plasma or serum or whole blood sample from the subject. In some embodiments of this treatment method, the neurodegenerative disorder being treated is Alzheimer's Disease (AD), Mild Cognitive Impairment (MCI), or Parkinson's Disease (PD).

In a fifth aspect, the present invention provides a method for assessing efficacy of a therapeutic agent for treating a neurodegenerative disorder in a subject. The method includes these steps: (1) comparing the subject's plasma or serum or whole blood level of Nell-1 protein before and after administration of the therapeutic agent to the subject; (2) detecting an increase in the subject's plasma or serum or whole blood level of Nell-1 protein after administration of the therapeutic agent; and (3) determining the therapeutic agent as effective for treating the neurodegenerative disorder. In some embodiments, the neurodegenerative disorder being treated is Alzheimer's Disease (AD), Mild Cognitive Impairment (MCI), or Parkinson's Disease (PD). In some embodiments, the claimed method further comprises, prior to step (1), measuring the plasma or serum or whole blood level of Nell-1 protein before and after administration. In some embodiments, the method further comprises, prior to the measuring step, obtaining a plasma or serum or whole blood sample from the subject before and after administration.

In some embodiments of the methods described above and herein, the subject or subjects being tested for a neurodegenerative disorder (such as AD, MCI, or PD) or its risk or being treated for a neurodegenerative disorder or its risk is/are of the Chinese descent.

### BRIEF DESCRIPTION OF THE DRAWINGS

FIG. 1. Decreased Nell-1 protein level in blood differentiates patients with AD from cognitively normal subjects. FIG. 1a: Box-and-whisker plot showing the individual plasma Nell-1 levels of cognitively normal (CN) subjects and patients with Alzheimer's disease (AD) from the Hong Kong Chinese cohort (n=119 CN+152 AD). Statistical analysis was performed by linear regression adjusting for age, sex, and history of cardiovascular disease; β=−0.158, *P<0.05. FIG. 1b: Receiver operating characteristic (ROC) curve and corresponding area under the curve (AUC) representing the performance of plasma Nell-1 level in classifying AD phenotype in the Hong Kong Chinese cohort.

FIG. 2. Decreased Nell-1 protein level in blood differentiates patients with MCI from cognitively normal subjects. FIG. 2a: Box-and-whisker plot showing the individual plasma Nell-1 levels of cognitively normal (CN) subjects and patients with mild cognitive impairment (MCI) from the Hong Kong Chinese cohort (n=119 CN+109 MCI). Statistical analysis was performed by linear regression adjusting for age, sex, and history of cardiovascular disease; β=−0.187, *P<0.05. FIG. 2b: Receiver operating characteristic (ROC) curve and corresponding area under the curve (AUC) representing the performance of plasma Nell-1 level in classifying MCI phenotype in the Hong Kong Chinese cohort.

FIG. 3. Decreased Nell-1 protein level in blood differentiates patients with PD from cognitively normal subjects. FIG. 3a: Box-and-whisker plot showing the individual plasma Nell-1 levels of cognitively normal (CN) subjects and patients with Parkinson's disease (PD) from the Hong Kong Chinese cohort (n=119 CN+39 PD). Statistical analysis was performed by linear regression adjusting for age and sex; β=−0.370, ***P<0.001. FIG. 3b: Receiver operating characteristic (ROC) curve and corresponding area under the curve (AUC) representing the performance of plasma Nell-1 level in classifying PD phenotype in the Hong Kong Chinese cohort.

FIG. 4. Decreased Nell-1 protein level in blood predicts neurodegeneration, AD progression, cognitive decline, and aging. FIG. 4a: Scatter plot and regression line (red) showing the association between plasma Nell-1 level and neurodegeneration indicated by plasma NfL level in the Hong Kong Chinese cohort (n=315). Statistical analysis was performed by linear regression adjusting for age, sex, and disease diagnosis; β=−0.005. FIG. 4b: Scatter plot and regression line (red) showing the association between plasma Nell-1 level and Alzheimer's disease (AD) progression indicated by plasma pTau181 level in the Hong Kong Chinese cohort (n=300). Statistical analysis was performed by linear regression adjusting for age, sex, and disease diagnosis; β=−0.043. FIG. 4c: Scatter plot and regression line (red) showing the association between plasma Nell-1 level and cognitive abilities indicated by MOCA score in the Hong Kong Chinese cohort (n=384). Statistical analysis was performed by linear regression adjusting for sex, years of education and disease diagnosis; β=0.012. FIG. 4d: Scatter plot and regression line (red) showing the association between plasma Nell-1 level and age in the Hong Kong Chinese cohort (n=424). Statistical analysis was performed by linear regression adjusting for sex and disease diagnosis; β=−0.014. R², Pearson's correlation coefficient.

FIG. 5. Mendelian randomization analysis identifies decreased Nell-1 protein level in blood as a causal factor of neurodegeneration. FIG. 5a: Manhattan plot showing the genetic variants associated with plasma Nell-1 levels in the Hong Kong Chinese cohort. The x-axis indicates the genetic variants position on chromosome 11 and the y-axis indicates the significance of the association. The dashed red line represents the significance threshold (P=0.01). Statistical analysis was performed by linear regression adjusting for age, sex, disease diagnosis, and population structure. cM/Mb, centimorgans per megabase. FIG. 5b: Scatter plot and regression line (blue) showing the causal association between plasma Nell-1 level and plasma NfL level in the Hong Kong Chinese cohort (n=255). Statistical analysis was performed by two-stage least-square regression adjusting for age, sex, disease diagnosis, and population structure.

FIG. 6. Treatment with Nell-1 protein promotes synapse formation in hippocampal neurons. Immunohistochemical analysis of excitatory synapse density in cultured primary rat hippocampal neurons treated with Nell-1 (2 μg/mL, 48 h) or DPBS (Con). FIG. 6a: Immunostaining of VGluT1 (red; presynaptic marker), PSD-95 (green; postsynaptic marker), and MAP2 (blue, dendrite marker). (Scale bar, 20 μm). FIG. 6b: Higher-magnification images of dendrites (white rectangles in a) showing excitatory synapses (VGluT1-positive PSD-95 cluster; white arrowheads). (Scale bar, 5 μm). FIG. 6c: Bar plots showing the quantification of VGluT1-positive PSD-95 clusters. (Statistical analysis performed by two-tailed unpaired t test, ***P<0.001; Con: n=15 neurons; Nell-1: n=14 neurons; two independent experiments).

FIG. 7. Treatment with Nell-1 protein rescues impaired hippocampal synaptic plasticity in APP/PS1 AD transgenic model mice. Electrophysiological analysis of long-term potentiation (LTP) in 11-month-old wildtype (WT) and APP/PS1 mice treated with Nell-1 (5 ng per day through intracerebroventricular delivery, 7 days) or artificial cerebrospinal fluid. LTP in the hippocampal CA1 region is induced by 3× high-frequency stimulation (HFS). FIG. 7a: Averaged slopes of baseline normalized field excitatory postsynaptic potential (fEPSP). FIG. 7b: Bar chart showing the normalized fEPSP slope 60 min after HFS stimulation. (Statistical analysis performed by one-way ANOVA with Tukey's post hoc test, *P<0.05; WT Con: n=10 brain slices from 4 mice; WT Nell-1: n=14 brain slices from 6 mice; APP/PS1 Con: n=10 brain slices from 5 mice; APP/PS1 Nell-1: n=11 brain slices from 4 mice, two independent experiments). Data are shown as mean±SEM.

FIG. 8. Treatment with Nell-1 protein increases hippocampal neurogenesis in mice. Immunohistochemical analysis of immature neurons in hippocampal dentate gyrus (DG) region in 11-month-old wild-type (WT) mice treated with Nell-1 (5 ng per day through intracerebroventricular delivery, 7 days) or artificial cerebrospinal fluid. FIG. 8a: Immunostaining of DCX (cyan; immature neuronal marker; Scale bar: 100 μm). FIG. 8b: Higher-magnification images of the DG region (white rectangles in a) showing immature neurons (white arrowheads; Scale bar: 50 μm). FIG. 8c: Bar chart showing the quantification of immature neurons in the DG subgranular zone. (Statistical analysis performed by two-tailed unpaired t-test, *P<0.05; Con: n=3 mice; Nell-1: n=3 mice).

### DEFINITIONS

As used herein, the term “Nell-1” refers to Protein kinase C-binding protein NELL1, also known as Nel-related protein 1 or Neural epidermal growth factor-like 1 protein. One exemplary Nell-1 protein has UniProtKB accession number Q92832, but the term “Nell-1” also encompasses all variants to this exemplary sequence.
The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19: 5081 (1991); Ohtsuka et al., J. Biol. Chem. 260: 2605-2608 (1985); and Rossolini et al., Mol. Cell. Probes 8: 91-98 (1994)). The term nucleic acid is used to encompass the subject matter as defined by the terms gene, cDNA, and mRNA encoded by a gene.
The term “gene” means the segment of DNA involved in producing a polypeptide chain. It may include regions preceding and following the coding region (leader and trailer) as well as intervening sequences (introns) between individual coding segments (exons).
“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.
In this disclosure the term “biological sample” or “sample” includes sections of tissues such as biopsy and autopsy samples, and frozen sections taken for histologic purposes, or processed forms of any of such samples. Biological samples include blood and blood fractions or products (e.g., whole blood, acellular fraction of blood (serum, plasma), and blood cells), sputum or saliva, lymph and tongue tissue, cultured cells, e.g., primary cultures, explants, and transformed cells, stool, urine, stomach biopsy tissue etc. A biological sample is typically obtained from a eukaryotic organism, which may be a mammal, may be a primate and may be a human subject.
The term “immunoglobulin” or “antibody” (used interchangeably herein) refers to an antigen-binding protein having a basic four-polypeptide chain structure consisting of two heavy and two light chains, said chains being stabilized, for example, by interchain disulfide bonds, which has the ability to specifically bind antigen. Both heavy and light chains are folded into domains.
The term “antibody” also refers to antigen- and epitope-binding fragments of antibodies, e.g., Fab fragments, that can be used in immunological affinity assays. There are a number of well characterized antibody fragments. Thus, for example, pepsin digests an antibody C-terminal to the disulfide linkages in the hinge region to produce F(ab)′₂, a dimer of Fab which itself is a light chain joined to V_{H}-C_{H}1 by a disulfide bond. The F(ab)′₂ can be reduced under mild conditions to break the disulfide linkage in the hinge region thereby converting the (Fab′)₂ dimer into an Fab′ monomer. The Fab′ monomer is essentially a Fab with part of the hinge region (see, e.g., Fundamental Immunology, Paul, ed., Raven Press, N.Y. (1993), for a more detailed description of other antibody fragments). While various antibody fragments are defined in terms of the digestion of an intact antibody, one of skill will appreciate that fragments can be synthesized de novo either chemically or by utilizing recombinant DNA methodology. Thus, the term antibody also includes antibody fragments either produced by the modification of whole antibodies or synthesized using recombinant DNA methodologies.
The phrase “specifically binds,” when used in the context of describing a binding relationship of a particular molecule to a protein or peptide, refers to a binding reaction that is determinative of the presence of the protein in a heterogeneous population of proteins and other biologics. Thus, under designated binding assay conditions, the specified binding agent (e.g., an antibody) binds to a particular protein at least two times the background and does not substantially bind in a significant amount to other proteins present in the sample. Specific binding of an antibody under such conditions may require an antibody that is selected for its specificity for a particular protein or a protein but not its similar “sister” proteins. A variety of immunoassay formats may be used to select antibodies specifically immunoreactive with a particular protein or in a particular form. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective binding reaction will be at least twice background signal or noise and more typically more than 10 to 100 times background. On the other hand, the term “specifically bind” when used in the context of referring to a polynucleotide sequence forming a double-stranded complex with another polynucleotide sequence describes “polynucleotide hybridization” based on the Watson-Crick base-pairing, as in the context of a “polynucleotide hybridization method.”
As used in this application, an “increase” or a “decrease” refers to a detectable positive or negative change in quantity from a comparison control, e.g., an established standard control (such as an average level/amount of a particular protein found in samples from healthy subjects who has not been diagnosed with a neurodegenerative disorder such as AD, MCI, or PD and has no increased risk for such disorder). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.
A “label,” “detectable label,” or “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radioactive component into the protein or used to detect antibodies specifically reactive with the protein. Typically a detectable label is attached to a probe or a molecule with defined binding characteristics (e.g., an antibody with a known binding specificity to a polypeptide antigen), so as to allow the presence of the probe (and therefore its binding target) to be readily detectable.
The term “amount” as used in this application refers to the quantity of a substance of interest, such as a polypeptide of interest, present in a sample. Such quantity may be expressed in the absolute terms, i.e., the total quantity of the substance in the sample, or in the relative terms, i.e., the concentration of the substance in the sample.
The term “subject” or “subject in need of treatment,” as used herein, includes individuals who seek medical attention due to risk of (e.g., with family history), or having been diagnosed of, a disease or condition, such as a neurodegenerative disorder such AD, MCI, or PD. Subjects also include individuals currently undergoing therapy that seek manipulation of the therapeutic regimen. Subjects or individuals in need of treatment include those that demonstrate symptoms of the neurodegenerative disorder, e.g., AD, or are at risk of suffering from the neurodegenerative disorder or its symptoms. For example, a subject in need of treatment includes individuals with a genetic predisposition or family history for the neurodegenerative disorder, those that have suffered relevant symptoms in the past, those that have been exposed to a triggering substance or event, as well as those suffering from chronic or acute symptoms of the condition. A “subject in need of treatment” may be at any age of life.
“Inhibitors,” “activators,” and “modulators” of a target protein are used to refer to inhibitory, activating, or modulating molecules, respectively, identified using in vitro and in vivo assays for the protein binding or signaling, e.g., ligands, agonists, antagonists, and their homologs and mimetics. The term “modulator” includes inhibitors and activators. Inhibitors are agents that, e.g., partially or totally block, decrease, prevent, delay activation, inactivate, desensitize, or down regulate the activity of the target protein. In some cases, the inhibitor directly or indirectly binds to the protein, such as a neutralizing antibody. Inhibitors, as used herein, are synonymous with inactivators and antagonists. Activators or agonists are agents that, e.g., stimulate, increase, facilitate, enhance activation, sensitize or up-regulate the activity of the target protein. Modulators include the target protein's ligands or binding partners, including modifications of naturally-occurring ligands and synthetically-designed ligands, antibodies and antibody fragments, antagonists, agonists, small molecules including carbohydrate-containing molecules, siRNAs, RNA aptamers, and the like.
The term “treat” or “treating,” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition.
The term “effective amount,” as used herein, refers to an amount that produces therapeutic effects for which a substance is administered. The effects include the prevention, correction, or inhibition of progression of the symptoms of a disease/condition and related complications to any detectable extent. The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).
A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.
The term “excipient” refers to any essentially accessory substance that may be present in the finished dosage form of the composition of this invention. For example, the term “excipient” includes vehicles, binders, disintegrants, fillers (diluents), lubricants, glidants (flow enhancers), compression aids, colors, sweeteners, preservatives, suspending/dispersing agents, film formers/coatings, flavors and printing inks.
The term “consisting essentially of,” when used in the context of describing a composition containing an active ingredient or multiple active ingredients, refer to the fact that the composition does not contain other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agent(s) effective for enhancing the expression or activity of Nell-1 protein in a subject is a composition that does not contain any other agents that may have any detectable positive or negative effect on the same target process (expression or activity of Nell-1 protein) or that may increase or decrease to any measurable extent of the disease occurrence or symptoms of a neurodegenerative disorder such as AD, MCI, or PD among the receiving subjects.
The term “about” denotes a range of +/−10% of a pre-determined value. For example, “about 10” sets a range of 90% to 110% of 10, i.e., 9 to 11.
The term “standard control,” as used herein, refers to a sample comprising an analyte of a predetermined amount to indicate the quantity or concentration of this analyte present in this type of sample (e.g., a predetermined DNA/mRNA or protein) taken from an average healthy subject not suffering from or at risk of developing a predetermined disease or condition (e.g., a neurodegenerative disorder such as AD, MCI, or PD). When used in the context of describing a value, this term may also be used to simply refer to the quantity or concentration of this analyte present in a “standard control” sample.
The term “average,” as used in the context of describing a healthy subject who does not suffer from and is not at risk of developing a relevant disease or disorders (for example, a neurodegenerative disorder such as AD, MCI, or PD) refers to certain characteristics, such as the level of a pertinent protein in the person's sample (e.g., serum or plasma or whole blood), that are representative of a randomly selected group of healthy humans who are not suffering from and is not at risk of developing the disease or disorder. This selected group should comprise a sufficient number of human subjects such that the average amount or concentration of the analyte of interest among these individuals reflects, with reasonable accuracy, the corresponding profile in the general population of healthy people. Optionally, the selected group of subjects may be chosen to have a similar background to that of a person whose is tested for indication or risk of the relevant disease or disorder, for example, matching or comparable age, gender, ethnicity, and medical history, etc.
The term “inhibiting” or “inhibition,” as used herein, refers to any detectable negative effect on a target biological process or on the level of a biomarker (e.g., a protein). Typically, an inhibition is reflected in a decrease of at least 10%, 20%, 30%, 40%, or 50% in one or more parameters indicative of the biological process or its downstream effect or the level of biomarker when compared to a control where no such inhibition is present. The term “enhancing” or “enhancement” is defined in a similar manner, except for indicating a positive effect, i.e., the positive change is at least 10%, 20%, 30%, 40%, 50%, 80%, 100%, 200%, 300% or even more in comparison with a control. The terms “inhibitor” and “enhancer” are used to describe an agent that exhibits inhibiting or enhancing effects as described above, respectively. Also used in a similar fashion in this disclosure are the terms “increase,” “decrease,” “more,” and “less,” which are meant to indicate positive changes in one or more predetermined parameters by at least 10%, 20%, 30%, 40%, 50%, 80%, 100%, 200%, 300% or even more, or negative changes of at least 10%, 20%, 30%, 40%, 50%, 80% or even more in one or more predetermined parameters.
As used herein, the term “Chinese” refers to ethnic Chinese people who and whose ancestors have been residing in the historical territories of China, including the mainland and Hong Kong, for a length of time, e.g., at least the last 3, 4, 5, 6, 7, or 8 generations or the last 100, 150, 200, 250, or 300 years.

### DETAILED DESCRIPTION OF THE INVENTION

### I. Introduction

Alzheimer' disease (AD) is one of the most common forms of dementia in the world, accounting for 60-70% of all dementia cases. It is an irreversible degenerative brain disease and a leading cause of mortality among the elderly. The hallmarks of this disease are deposition of extracellular β-amyloid (Aβ) plaques and intracellular neurofibrillary tangles, which result in declining memory, reasoning, judgment, and locomotion abilities, with symptoms worsening over time.

Currently, an estimated 55 million people worldwide are afflicted with dementia, among which 60-80% suffer from AD. This figure is expected to rise significantly to 153 million by 2050 due to longer life expectancies. There is no cure for AD, and the pathophysiology of the disease is still relatively unknown. Currently, there are only six drugs approved by the US Food and Drug Administration (FDA) to treat AD, but these at best alleviate AD symptoms only rather than address disease pathology, as they cannot reverse the condition or prevent further deterioration, and they are ineffective for treating severe conditions. Thus, early diagnosis and early therapeutic intervention is critical in the management of AD. Research has confirmed that AD affects the brain long before actual symptoms of memory loss or cognitive decline actually manifest. To this date, however, there are no effective and reliable diagnostic tools for early detection of AD. By the time a patient is diagnosed with AD using standard methods currently in use, which involves subjective clinical assessment, the pathological symptoms are already at an advanced stage. The present disclosure provides diagnostic methods utilizing protein marker Nell-1 (also known as Protein kinase C-binding protein NELL1, Nel-related protein 1, or Neural epidermal growth factor-like 1 protein) for diagnosing the presence of a neurodegenerative disorder, such as AD, MCI, or PD, or for assessing one's risk of later developing the neurodegenerative disorder, so as to allow early diagnosis and to enable early treatment remedies aiming at preserving/restoring one's cognitive ability while using Nell-1 protein as a therapeutic target and a surrogate indicative of disease status.

### II. Quantitation of Marker Protein

### A. Obtaining Samples

The first step of practicing the present invention is to obtain a blood sample from a subject being tested for assessing the risk of developing a neurodegenerative disorder (such as AD, MCI, or PD) or monitoring for the neurodegenerative disorder's severity or progression. Samples of the same type should be taken from both a control group (cognitively normal individuals not suffering from the neurodegenerative disorder and without increased risk for the neurodegenerative disorder) and a test group (subjects being tested for possible neurodegenerative disorder such AD, MCI, or PD or for increased risk for the neurodegenerative disorder, for example). Standard procedures routinely employed in hospitals or clinics are typically followed for this purpose.

For the purpose of detecting the presence/quantity of marker proteins or assessing the risk of developing a neurodegenerative disorder in test subjects, individual patients' blood samples are taken, and the serum/plasma or whole blood level of a pertinent marker protein (e.g., Nell-1 protein) may be measured and then compared to a standard control. If a decrease in the level of Nell-1 protein (e.g., after being normalized) is observed when compared to the control level, the test subject is deemed to have a neurodegenerative disorder such as AD, MCI, or PD or have an elevated risk of developing later developing the condition. For the purpose of monitoring disease progression or assessing therapeutic effectiveness in neurodegenerative disorder patients, individual patient's blood samples may be taken at different time points, such that the level of Nell-1 protein can be measured to provide information indicating the state of disease. For instance, when a patient's Nell-1 protein level shows a general trend of increasing over time, the patient is deemed to be improving in the severity of the neurodegenerative disorder or the therapy the patient has been receiving is deemed effective for treating the disorder. A lack of substantial change or a continued decrease in a patient's Nell-1 protein level would indicate a lack of change in the status of the neurodegenerative disorder and ineffectiveness of the therapy given to the patient.

Moreover, the present inventors have devised methods to (1) assess the relative risk for developing a neurodegenerative disorder such as AD, MCI, or PD, or (2) assess the relative severity of the neurodegenerative disorder such as AD, MCI, or PD, between two or among three or more individuals based on their relative levels of Nell-1 protein in their plasma/serum or blood samples. The lower the Nell-1 protein level, the higher the risk or severity of the neurodegenerative disorder in an individual compared to others.

### B. Preparing Samples for Protein Detection

The blood sample from a subject is suitable for the present invention and can be obtained by well-known methods and as described in standard medical literature. In certain applications of this invention, serum or plasma or whole blood may be the preferred sample type. In other cases, whole blood samples may be used.

A blood sample is obtained from a person to be tested, assessed, or monitored for a neurodegenerative disorder using a method of the present invention. Collection of blood sample from an individual is performed in accordance with the standard protocol hospitals or clinics generally follow. An appropriate amount of blood is collected and may be stored according to standard procedures prior to further preparation.

The analysis of a marker protein (e.g., Nell-1 protein) found in a patient's sample according to the present invention may be performed using, e.g., serum or plasma or whole blood. The methods for preparing patient samples for protein extraction/quantitative detection are well known among those of skill in the art.

### C. Determining the Level of Marker Protein

A protein of any particular identity, such as Nell-1 protein, can be detected using a variety of immunological assays. In some embodiments, a sandwich assay can be performed by capturing the protein from a test sample with an antibody having specific binding affinity for the protein. The protein then can be detected with a labeled antibody having specific binding affinity for it. Such immunological assays can be carried out using microfluidic devices such as microarray protein chips. A protein of interest (e.g., Nell-1 protein) can also be detected by gel electrophoresis (such as 2-dimensional gel electrophoresis) and western blot analysis using specific antibodies. Alternatively, standard immunohistochemical techniques can be used to detect a given protein (e.g., Nell-1 protein), using the appropriate antibodies. Both monoclonal and polyclonal antibodies (including antibody fragment with desired binding specificity) can be used for specific detection of the polypeptide. Such antibodies and their binding fragments with specific binding affinity to a particular protein (e.g., Nell-1 protein) can be generated by known techniques.

Other methods may also be employed for measuring the level of a marker protein in practicing the present invention. For instance, a variety of methods have been developed based on the mass spectrometry technology to rapidly and accurately quantify target proteins even in a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8 (2): 787-797.

### III. Establishing a Standard Control

In order to establish a standard control for practicing the method of this invention, a group of healthy persons free of a neurodegenerative disorder (such as AD, MCI, or PD) or increased risk for developing the neurodegenerative disorder as conventionally defined is first selected. These individuals are within the appropriate parameters, if applicable, for the purpose of screening for and/or monitoring a neurodegenerative disorder using the methods of the present invention. Optionally, the individuals are of same gender, similar age, or similar ethnic background to the test subjects.

The healthy status of the selected individuals is confirmed by well-established, routinely-employed methods including but not limited to general physical examination of the individuals and general review of their medical history.

Furthermore, the selected group of healthy individuals must be of a reasonable size, such that the average amount/concentration of the marker protein (e.g., Nell-1 protein) in the serum or plasma or whole blood sample obtained from the group can be reasonably regarded as representative of the normal or average level among the general population of healthy people without a neurodegenerative disorder (such as AD, MCI, or PD) or increased risk for the neurodegenerative disorder. Preferably, the selected group comprises at least 10, 20, 30, or 50 human subjects.

Once an average value for the marker protein is established based on the individual values found in each subject of the selected healthy control group, this average or median or representative value or profile is considered a standard control. A standard deviation is also determined during the same process. In some cases, separate standard controls may be established for separately defined groups having distinct characteristics such as age, gender, or ethnic background.

### IV. Monitoring and Treatment

### A. Treatment by Known Remedies

In a related aspect, the present invention also supports and enables treatment for a neurodegenerative disorder (such as AD, MCI, or PD) upon detection of the presence of the neurodegenerative disorder, or a heightened risk of later developing the neurodegenerative disorder, or a worsening condition of the neurodegenerative disorder in a patient. In some embodiments, the method comprises, upon determining a subject as having an increased risk for AD, administering a treatment to said subject, for example, antibody drugs such as lecanemab, acetylcholinesterase inhibitors (such as donepezil, galantamine, rivastigmine), memantine, glutamate receptor blockers, citalopram, fluoxetine, paroxeine, sertraline, trazodone, lorazepam, oxazepam, aripiprazole, clozapine, haloperidol, olanzapine, quetiapine, risperidone, ziprasidone, nortriptyline, tricyclic antidepressants, benzodiazepines, temazepam, zolpidem, zaleplon, chloral hydrate, coenzyme Q10, ubiquinone, coral calcium, Ginkgo biloba, huperzine A, omega-3 fatty acids, phosphatidylserine, or any combination thereof.

In some cases, when the diagnostic method steps described above and herein are completed, optionally with additional diagnostic examination performed to provide further confirmatory information (for example, by brain imaging via CT scan or other imaging techniques to show excessive loss of brain volume, or by testing cognitive capability to show an accelerated decline), and a patient has been determined to either already have AD or is at a significantly increased risk of later developing AD, suitable therapeutic or prophylactic regimens may be ordered by physicians or other medical professionals to treat the patient, to manage/alleviate the ongoing symptoms, or to delay the future onset of the disease. The U.S. Food and Drug Administration (FDA) has approved a number of cholinesterase inhibitors, including donepezil (Aricept™, the only cholinesterase inhibitor approved to treat all stages of AD, including moderate to severe), rivastigmine (Exelon™, approved to treat mild to moderate AD), galantamine (Razadyne™, mild to moderate patients) and memantine (Namenda™). Donepezil is the only cholinesterase inhibitor approved to treat all stages of AD, including moderate to severe. Any one or more of these drugs can be prescribed for treating patients who have been diagnosed with AD in accordance with the methods of this invention. Also, Biogen's antibody drugs aducanumab and lecanemab recently received full approval by the FDA. Another possibility of treatment is administration of trazodone, which is currently approved for use as an antidepressant and has been reported as an effective agent for ameliorating AD symptoms.

For patients who are deemed at a high or an increased risk for developing a neurodegenerative disorder, such as AD, MCI, or PD, in a future time but do not yet exhibit any clinical symptoms, continuous monitoring is also appropriate, especially at an increased frequency. For example, the patients may be subject to more frequently scheduled regular testing (e.g., once every six months, once a year, or once every two years) to detect any accelerated change in their cognitive capabilities. Methods suitable for such regular monitoring include General Practitioner Assessment of Cognition (GPCOG), Mini-Cog, Eight-item Informant Interview to Differentiate Aging and Dementia (AD8), and Short Informant Questionnaire on Cognitive Decline in the Elderly (IQCODE). Furthermore, prophylactic treatment with trazodone may also be recommended.

For patients who have been diagnosed according to the methods of this invention as already suffering from MCI or PD or as having an increased risk of later developing MCI or PD, various medications may be employed to treat these patients, in both therapeutic and prophylactic contexts. For example, patients diagnosed with MCI or at risk of MCI can be treated with certain AD drugs, e.g., aducanumab or lecanemab, or by stoppage of ongoing administration of certain medications, e.g., benzodiazepines (used to treat conditions such as anxiety, seizures and sleep disturbances), anticholinergics (which affect chemicals in the nervous system to treat many different types of conditions), antihistamines (used to manage allergy symptoms), opioids (used to treat pain), and proton pump inhibitors (used to treat reflux or gastroesophageal reflux disease, or GERD), or by administration of medications for treating certain medical conditions (such as high blood pressure, depression, and sleep apnea) known to contribute to/exacerbate MCI symptoms. In addition, modifications to patients' life style and social behavior are made for alleviating MCI symptoms or slowing cognitive decline or reducing MCI risks, including regular physical exercise, adopting a diet low in fat and rich in fruits and vegetables, supplementation of omega-3 fatty acids, engagement in a mentally and socially active life style, up to professionally administered memory training and other cognitive training programs.

For patients diagnosed with PD or at risk of PD, administration of medications such as carbidopa-levodopa (including inhalation and infusion versions), Duopa, dopamine agonists (e.g., pramipexole (Mirapex ER), rotigotine (Neupro), and dpomorphine (Apokyn)), monoamine oxidase B (MAO B) inhibitors (e.g., selegiline (Zelapar), rasagiline (Azilect), and safinamide (Xadago)), catechol O-methyltransferase (COMT) inhibitors (e.g., entacapone (Comtan), opicapone (Ongentys), and Tolcapone (Tasmar)), anticholinergics, amantadine, adenosine receptor antagonists (A2A receptor antagonists such as istradefylline (Nourianz)), and nuplazid (Pimavanserin) might be appropriate. Life style and home remedies including regular exercise and healthy eating can also be employed to treat PD patients and individuals at risk of PD.

### B. Treatment by Targeting Nell-1

In addition to the use of known therapeutic agents and remedies for the treatment of a neurodegenerative disorder, such as AD, MCI, or PD, the discovery made by the present inventors, namely the correlation between a depressed circulating Nell-1 protein level and the presence, increased risk, or severity of a neurodegenerative disorder such as AD, MCI, or PD, enables novel and effective means for treating such neurodegenerative disorder applicable in both prophylactic and therapeutic contexts.

More specifically, this invention provides therapeutic use of Nell-1 protein or nucleic acid encoding the protein for treating the neurodegenerative disorder including AD, MCI, and PD by enhancing the Nell-1 protein levels, especially in the patient's brain. For instance, patients may be directly administered with a composition comprising an effective amount of a recombinant Nell-1 protein via suitable administration routes and delivery systems. In other cases, the rate of Nell-1 protein synthesis may also be increased by modulating the NELL1 gene expression at the cellular source using genetic engineering techniques (e.g., Nell-1 gene knock-in by a CRISPR-based system), with a focus on pertinent target sites (e.g., brain). Moreover, the treatment may be achieved by activating the downstream signaling pathways of Nell-1 by stimulating the Nell-1-specific receptor, Cntnap4, using one or more appropriate agonists, including agonistic antibodies for Cntnap4, small molecules, or peptides, such as certain peptide fragments of Nell-1 (see, e.g., Li et al., J Bone M Res, 33 (10), 1813-1825, 2018). Because Cntnap4 is predominantly expressed in the brain, a pharmaceutical composition comprising such an agonist is preferably delivered using suitable delivery vehicles that can permeate the blood-brain barrier while specifically targeting the brain.

The present invention provides pharmaceutical compositions comprising or consisting essentially of an effective amount of an active therapeutic agent (e.g., Nell-1 protein or a nucleic acid encoding the Nell-1 protein, a Nell-1 receptor protein or a nucleic acid encoding the receptor, or an agonist/activator of Nell-1 protein or its receptor) and one or more physiologically or pharmaceutically acceptable excipients. Pharmaceutical compositions of the invention are suitable for use in a variety of drug delivery systems. Suitable formulations for use in the present invention are found, e.g., in Remington's Pharmaceutical Sciences, Mack Publishing Company, Philadelphia, PA, 17th ed. (1985). For a brief review of methods for drug delivery, see, Langer, Science 249: 1527-1533 (1990).

The pharmaceutical compositions of the present invention can be administered by various routes, e.g., systemic administration via intravenous, intramuscular, or subcutaneous injection, as well as local delivery such as by intracranial or intraperitoneal injection. One route of administering the pharmaceutical compositions is intravenous administration at daily doses of about 1 to about 1000 μg, about 5 to about 500 μg, about 10 to about 250 μg, about 20 to about 100 μg, or about 25 to about 50 μg of the nucleic acid of this invention. In the alternative, the pharmaceutical compositions are administered via intravenous delivery at daily doses of about 1 to about 1000 mg, about 5 to about 500 mg, about 10 to about 250 mg, about 20 to about 100 mg, or about 25 to about 50 mg of a recombinant protein or an activator/agonist in accordance with the disclosure of this invention. Additionally, the composition may be formulated in a daily, weekly, or monthly dosage format for administration to the subject. The appropriate dose may be administered in a single, one-time daily dose or as divided doses presented at appropriate intervals, for example one dose every two, three, four, five, six, or more months such as every 12 months.

Polynucleotide sequences including RNA or any derivatives or modified versions thereof may be chemically synthesized according to methods known in the pertinent technical field. An RNA molecule can be modified by substitution with one or more nucleotide analogs and/or at the base moiety, sugar moiety, or phosphate backbone, for example, to improve stability of the molecule, hybridization or binding capability, or bioavailability, etc. The polynucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84:648-652; WO 88/09810) or the blood-brain barrier (see, e.g., WO 89/10134), hybridization-triggered cleavage agents (See, e.g., Krol et al., 1988, BioTechniques 6: 958-976) or intercalating agents (see, e.g., Zon, 1988, Pharm. Res. 5: 539-549).

In some embodiments, the composition comprising an active agent of this invention, e.g., a nucleic acid encoding Nell-1 protein, is formulated as a composition of nanoparticles, especially in the form of lipid nanoparticles (LNP) comprising the active agent, such as a nucleic acid, which may be in the form of DNA or RNA, including modified RNA. One or more types of lipid, as well as other ingredients, may be used in the formulation. For example, the LNP may comprise a cationic lipid, a neutral lipid, a steroid, a polymer conjugated lipid, and the active agent (e.g., a nucleic acid such as DNA or RNA, including their chemically modified version). In some cases, the LNP may further comprise at least one lipid or lipid-like material other than a cationic or cationically ionizable lipid or lipid-like material, at least one polymer other than a cationic polymer, or a mixture thereof. The preferred mode of administration of DNA- or RNA-encapsulated LNP compositions is intravenous or intracranial administration, for example, a preservative-free, sterile dispersion of nucleic acid formulated in lipid nanoparticles in aqueous cryoprotectant buffer for local or systemic injection. In some embodiments, a LNP formulation carrying an active agent of this invention is used to facilitate the effective crossing of the blood-brain barrier, optionally with a suitable affinity moiety specifically targeting brain cells (e.g., a binding partner to a brain cell surface receptor) conjugated with the lipid component on the outer surface of the nanoparticles to further enhance brain-specific delivery of the active agent.

Moreover, gene editing technologies can be used in the therapeutic scheme of this invention. For example, the CRISPR/Cas system have been adapted for use in targeted gene editing in eukaryotic cells. See, e.g., Ledford (2016), Nature 531 (7593): 156-9. Additional gene editing systems that can be used for practicing the present invention include TALENs (Transcription activator-like effector nucleases), ZFNs (Zinc-finger nucleases), and base editing, as well as newly developed techniques such as homing endonucleases and meganucleases (MegNs) (which target and cleave DNA sequences) and prime editing (which generates RNA templates for gene alteration).

As illustrated in this disclosure, a lower than normal Nell-1 protein level in a patient's blood/serum/plasma sample indicates the presence of or an increased risk for a neurodegenerative disorder such as AD, MCI, or PD in the patient, whereas a lower Nell-1 protein level corresponds to a worsened or further deteriorated disease state of the neurodegenerative disorder. Thus, to assess the effectiveness of a therapeutic regime, a patient's Nell-1 protein level found in a blood sample (e.g., a plasma or serum sample) is measured after the administration of a therapeutic composition comprising an effective amount of an active agent of this invention (e.g., Nell-1 protein or a nucleic acid encoding the Nell-1 protein, a Nell-1 receptor protein or a nucleic acid encoding the receptor, or an agonist/activator of Nell-1 protein or its receptor) over a reasonable time period, preferably both before and after the administration. Therapeutic efficacy is indicated by an increase in Nell-1 protein level in a blood/serum/plasma sample from a patient post-treatment in comparison to the pre-treatment level found in the same type of blood sample from the patient, or by a finding that Nell-1 protein level in a blood/serum/plasma sample from a patient post-treatment is no lower than a standard control value expected in the same type of blood sample from an average cognitively normal individual.

### V. Kits and Devices

The invention provides compositions and kits for practicing the methods described herein to assess marker protein Nell-1 level in a subject's serum/plasma or whole blood, which can be used for various purposes such as detecting or diagnosing the presence of a neurodegenerative disorder (such as AD, MCI, or PD), determining the risk of developing the neurodegenerative disorder, and monitoring progression of the neurodegenerative disorder in a patient, including assessing the therapeutic efficacy of a therapy administered for the neurodegenerative disorder among patients who have received a diagnosis of the disorder and have undergone treatment.

Kits for carrying out assays for determining marker protein levels typically include at least one antibody useful for specific binding to the Nell-1 protein amino acid sequence. Optionally, this antibody is labeled with a detectable moiety. The antibody can be either a monoclonal antibody or a polyclonal antibody. In some cases, the kits may include at least two different antibodies, one for specific binding to Nell-1 protein (i.e., the primary antibody) and the other for detection of the primary antibody (i.e., the secondary antibody), which is often attached to a detectable moiety.

Typically, the kits also include an appropriate standard control. The standard controls indicate the average value of marker protein Nell-1 in the serum or plasma or whole blood of healthy subjects who are not suffering from nor are at increased risk of developing a neurodegenerative disorder such as AD, MCI, or PD. In some cases, such standard control may be provided in the form of a set value. In addition, the kits of this invention may provide instruction manuals to guide users in analyzing test samples and assessing the presence or risk of the neurodegenerative disorder (such as AD, MCI, or PD), or assessing the disorder's status/severity/progression in a test subject.

In a further aspect, the present invention can also be embodied in a device or a system comprising one or more such devices, which is capable of carrying out all or some of the method steps described herein. For instance, in some cases, the device or system performs the following steps upon receiving a serum or plasma or whole blood sample taken from a subject being tested for detecting a neurodegenerative disorder (such as AD, MCI, or PD), assessing the risk of developing the neurodegenerative disorder, or assessing the disease severity/status/progression: (a) determining in sample the amount or concentration of marker protein Nell-1; (b) comparing the amount/concentration with a standard control value; and (c) providing an output indicating whether a neurodegenerative disorder (such as AD, MCI, or PD) is present in the subject or whether the subject is at increased risk of developing the neurodegenerative disorder, or whether the patient has a higher risk of later developing the neurodegenerative disorder relative to another patient being tested. In other cases, the device or system of the invention performs the task of steps (b) and (c), after step (a) has been performed and the amount or concentration from (a) has been entered into the device. Preferably, the device or system is partially or fully automated.

### EXAMPLES

The following examples are provided by way of illustration only and not by way of limitation. Those of skill in the art will readily recognize a variety of non-critical parameters that could be changed or modified to yield essentially the same or similar results.

### Introduction

Neurological disorders include a wide range of diseases of the nervous system. For instance, Alzheimer's disease (AD) is a neurodegenerative disorder characterized by progressive cognitive decline. AD is the leading cause of dementia and the 7^{th} leading cause of death globally. As of 2020, over 55 million people worldwide are afflicted with dementia, and the patient population is expected to reach 139 million by 2050 due to accelerated aging of the global population (Gauthier et al., World Alzheimer Report 2021: Journey through the diagnosis of dementia. London, England: Alzheimer's Disease International). Unfortunately, there currently is no effective treatment that can stop or reverse disease progression of AD.

The limited diagnostic measures for AD hinder the development of treatments. AD progression involves an extensive preclinical period, during which pathological changes occur in the brain with no manifestation of cognitive impairment. Identifying and correcting the disease-causing factors at the early stage of disease is desirable for effective treatment. However, the current diagnostic methods for AD (e.g., clinical assessment of cognitive functions, brain imaging, and detection of amyloid-beta and tau protein in the cerebrospinal fluid) are inconvenient, expensive, and often only detect AD in its late-stage. Therefore, a new diagnostic biomarker that can detect pathological changes in the brain at its early stage and is accessible to the public is urgently needed.

Several plasma proteins have shown to elicit regulatory effects on brain function, such as neurogenesis and microglial activation, by either directly permeating the blood-brain-barrier or indirectly via modulating activity of infiltrating immune cells. Moreover, many plasma proteins are deregulated in blood of patients with neurological diseases such as AD. Taken together, deregulated plasma proteins may serve as biomarkers to monitor disease progression or as therapeutic targets.

Here, Nell-1—a brain-derived growth factor-like protein—is identified as a promising diagnostic biomarker and therapeutic target for neurological diseases. Decreased plasma Nell-1 protein level can be used to differentiate patients with neurological diseases, such as AD, mild cognitive impairment (MCI), and Parkinson's disease (PD), from cognitively normal populations. Plasma Nell-1 level can also be used to monitor neurodegeneration and other related pathological changes in the brain. Moreover, elevated plasma Nell-1 has beneficial effects on the brain, including reduced neurodegeneration, enhanced synaptic functions, synaptic plasticity, and neurogenesis. Overall, Nell-1 is demonstrated not only as an effective blood biomarker for monitoring and classifying neurological diseases but also a therapeutic target to treat such diseases.

### Materials and Methods

### Subject Recruitment

Hong Kong Chinese individuals over the age of 60 years were recruited from the Special Outpatient Department of the Prince of Wales Hospital, the Chinese University of Hong Kong (n=119, 109, 152, and 39 for cognitively normal (CN) subjects, patient with MCI, patients with AD, and patients with PD, respectively). The clinical diagnoses were made based on the American Psychiatric Association's Diagnostic and Statistical Manual of Mental Disorders, Fifth Edition (DSM-5). This study was approved by the Prince of Wales Hospital of the Chinese University of Hong Kong as well as the Hong Kong University of Science and Technology. All participants provided written informed consent for both study participation and sample collection.

### Detection of Plasma Proteins

Blood samples were collected in EDTA tubes using standard protocols. Plasma was prepared by centrifugation and stored at −80 °C until use. The plasma levels of Nell-1 were measured by Olink Oncology III panel. The plasma levels of neurofilament light polypeptide (NfL) and phosphorylated-tau-181 (pTau181) were measured by the Simoa Neurology 4-Plex E Advantage Kit and P-Tau 181 Advantage V2 Kit, respectively.

### Whole-Genome Sequencing

DNA samples of participants were submitted to Novogene for library construction and WGS. Samples were sequenced on an Illumina Hiseq X (average depth: 5×). Genotype results stored in VCF files were used for principal component analysis. The top five principal components were generated by PLINK software.

### Analysis of the Association Between Plasma Proteins and Disease Phenotypes

Plasma Nell-1 levels were transformed by rank-based inverse normalization using the RankNorm function from the RNOmni package in R. The deregulation of plasma Nell-1 levels in patients with neurological diseases, such as MCI, AD, and PD, compared to control was determined using the association between normalized plasma Nell-1 level and disease phenotype, adjusting for age and sex. The following linear model was used (βᵢ, the weighted coefficients; ε, the intercept):
Normalized plasma Nell-1 level~β1 Disease phenotype+β2 Age+β3 Sex+ε

The relationship of plasma Nell-1 level and neurodegeneration-related endophenotypes, such as plasma NfL and pTau181 levels, was determined using the association between normalized plasma Nell-1 level and endophenotypes, adjusting for age and sex. The following linear model was used:
Normalized plasma Nell-1 level~β1 neurodegeneration-related endophenotype+β2 Age+β3 Sex+ε

### Valuation of Prediction Accuracy

The receiver operating characteristic (ROC) curves and areas under the curve (AUCs) of prediction models for MCI and AD risk prediction was generated using GraphPad Prism (version 8.2.0). The accuracy of the prediction model was denoted by the AUC values.

### One-Sample Mendelian Randomization

Association between plasma Nell-1 levels and all variants in the NELL1 gene locus (±1 Mb) was analyzed using linear regression adjusting for age, sex, disease phenotype, and the first five principal components of ancestry (n=108 and 147 for CN participants and patients with AD from the Hong Kong Chinese AD cohort). Association test was performed using PLINK (version 1.9). Genetic variants with minor allele frequency >1% and Hardy-Weinberg Equilibrium P>5×10⁻⁶ were selected for analysis. Considering the limited sample size, genetic associations were considered significant if P<0.01. LD-based clumping was performed so that only the top variants (i.e., smallest P-value) of each clump was retained while variants linked to the top variants were removed (R²<0.2). Finally, 20 genetic variants were deemed suitable to be used as instrumental variables for Mendelian randomization (MR) analysis (i.e., rs10741830, rs10833529, rs10833718, rs11025266, rs11026285, rs12795698, rs138024111, rs140262672, rs147617279, rs148361725, rs17840362, rs3045385, rs4923193, rs61155013, rs7109477, rs7112107, rs75540817, rs77814312, rs7938616, and rs867221555).

In MR, the causal association between an exposure (e.g., plasma Nell-1 level) and outcome (e.g., neurodegeneration-related endophenotypes) can be assessed using their association to instrumental variables. Here, one-sample MR analysis was performed using the two-stage least squares regression. In brief, each individual's predicted plasma Nell-1 level was calculated based on the genotypes of the instrumental variables. Then, the predicted values were regressed against neurodegeneration-associated endophenotypes (e.g., plasma NfL and pTau181 levels). The significance threshold was set as P<0.05.

### Culture of Rat Primary Hippocampal Neurons

Sprague-Dawley rat embryos were sacrificed on embryonic day 18, and the hippocampus was isolated and dissociated using trypsin. Hippocampal cells were seeded on 18-mm coverslips coated with 1 mg/mL poly-D-lysine at 0.3×105 cells per coverslip. Cultured cells were maintained in neurobasal medium (Invitrogen) supplemented with 2% B27 (Invitrogen) and 0.5 mM L-glutamine and incubated at 37 °C in a humidified atmosphere with 5% CO₂. To examine the role of Nell-1 in excitatory synapse formation, cultured hippocampal cells were treated with recombinant human Nell-1 (2 μg/mL; R&D Systems) or DPBS at 12-14 days in vitro (DIV).

### Immunostaining and Quantification of Cultured Rat Primary Hippocampal Neurons

Following the 48 hours treatment, cells were fixed with 4% paraformaldehyde/4% sucrose (wt/vol) and immunostained with VGLuT1 (AB5905, Sigma-Aldrich) and PSD-95 (ab18258, Abcam) antibodies. Cultures were then counterstained with DAPI and mounted in ProLong Diamond Antifade Mountant (P36961, Invitrogen). Images were acquired using a Leica TCS SP8 confocal system. To examine the changes in excitatory synapses in cultured hippocampal cells, VGluT1-positive PSD-95 clusters were quantified using ImageJ.

### Mice

All experiments involving mice were approved by the Hong Kong University of Science and Technology (HKUST) Animal Ethics Committee and conducted following the Guidelines of the Animal Care Facility of HKUST. All mice were housed in the HKUST Animal and Plant Care Facility with a 12-h light/dark cycle. The APP/PS1 transgenic mice were obtained from the Jackson Laboratory. Mice were randomly assigned to experimental conditions.

### In Vivo Experiments in Mice

Mini-osmotic pumps (model 1004; Alzet) were prepared with recombinant mouse NELL1 protein (7109-NL, R&D Systems; 189 ng per pump; 0.21 ng/h) or artificial cerebrospinal fluid as a control. The cannula of the pump was subsequently implanted into the brain ventricle for intracerebroventricular delivery. Mice were sacrificed after 7 days of treatment.

### Electrophysiology

Immediately after being sacrificed, the mice were dissected, and their brains were placed in ice-cold artificial cerebrospinal fluid oxygenated with 95% O₂/5% CO₂. Subsequently, the brains were sectioned into 300 μm slices using a vibrating blade microtome (VT1000S, Leica). The brain slices were placed on MED-P210A probe (Panasonic International), positioning the electrodes at the hippocampal region. The field excitatory postsynaptic potentials (fEPSPs) were recorded from the dendritic layer of the hippocampal CA1 neurons. The baseline stimulation intensity was selected as the intensity that elicited 30-40% of the maximum fEPSP response. Long-term potentiation (LTP) was induced by three trains of high-frequency stimulation (100 Hz for 1 s, delivered 30 s apart). After tetanus, the response was recorded for 1 h. LTP was quantified as the change in the average slope of the fEPSP over 1 h after LTP induction.

### Immunostaining of Mouse Brains

Mice were dissected and mouse brains were fixed in 4% paraformaldehyde at 4 °C for 24 h. Fixed brains were sectioned into 30 μm slices using a vibrating blade microtome (VT1200S, Leica). The sections were rinsed with DPBS and blocked with 4% goat serum, 1% BSA, 0.4% Triton X-100 and DPBS at room temperature for 1 h. The sections were then labeled with DCX antibody (AB2253, Sigma-Aldrich) at 4 °C overnight. The following day, the sections were labeled with fluorophore-conjugated anti-guinea pig secondary antibody at room temperature for 2 h. The sections were then labeled with the nuclear staining dye (DAPI) and mounted onto slides. Sections were imaged using the ZEISS LSM 980 with Airyscan 2.

### Statistical Analysis and Data Visualization

The investigators who performed the protein detection were blinded to the phenotypes of the human participants. The significance of association analyses was assessed by linear regression adjusting for age and sex. The level of significance was set at P<0.05. Statistical plots were generated using the ggplot function from the ggplot2 package in R or GraphPad Prism (version 8.2.0).

### Example I: Assessment of the AD Risk Using Nell-1 Protein Level in Blood

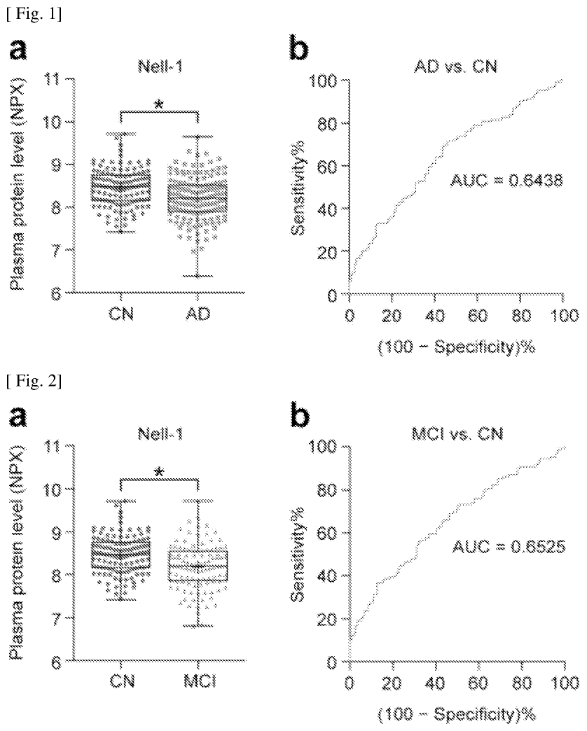

Nell-1 protein level was measured in plasma samples collected from the Hong Kong Chinese cohort (n=119 cognitively normal [CN] subjects and 152 patients with Alzheimer's disease [AD]). Plasma Nell-1 protein level was significantly decreased in patients with AD compared to CN subjects (β=−0.158, P<0.05; FIG. 1a). Accordingly, plasma Nell-1 protein level can be used to differentiate patients with AD from CN individuals with an accuracy of 64.38% (FIG. 1b). Moreover, having a lower plasma Nell-1 protein level can indicate a higher risk of developing AD. Taken together, Nell-1 protein level in blood can help assess the risk of developing AD.

### Example II: Assessment of the MCI Risk Using Nell-1 Protein Level in Blood

Nell-1 protein level was measured in plasma samples collected from the Hong Kong Chinese cohort (n=119 cognitively normal [CN] subjects and 109 patients with mild cognitive impairment [MCI]). Plasma Nell-1 protein level was significantly decreased in patients with MCI compared to CN subjects (β=−0.187, P<0.05; FIG. 2a). Accordingly, plasma Nell-1 protein level can be used to differentiate patients with MCI from CN individuals with an accuracy of 65.25% (FIG. 2b). Moreover, having a lower plasma Nell-1 protein level can indicate a higher risk of developing MCI. Taken together, Nell-1 protein level in blood can help assess the risk of developing MCI.

### Example III: Assessment of the PD Risk Using Nell-1 Protein Level in Blood

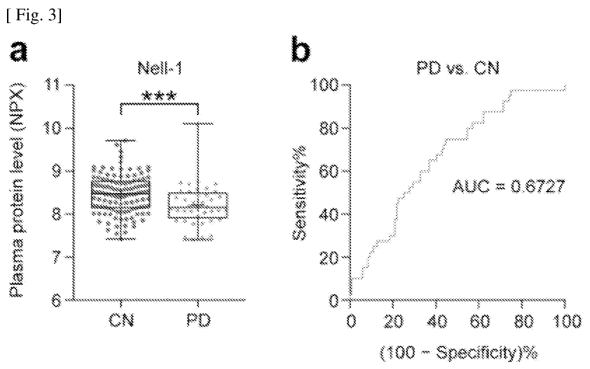

Nell-1 protein level was measured in plasma samples collected from the Hong Kong Chinese cohort (n=119 cognitively normal [CN] subjects and 39 patients with Parkinson's disease [PD]). Plasma Nell-1 protein level was significantly decreased in patients with PD compared to CN subjects (β=−0.370, P<0.001; FIG. 3a). Accordingly, plasma Nell-1 protein level can be used to differentiate patients with PD from CN individuals with an accuracy of 67.27% (FIG. 3b). Moreover, having a lower plasma Nell-1 protein level can indicate a higher risk of developing PD. Taken together, Nell-1 protein level in blood can help assess the risk of developing PD.

### Example IV: Assessment of the Level of Neurodegeneration, AD Progression, and Related Pathological Changes in the Brain Using Nell-1 Protein Level in Blood

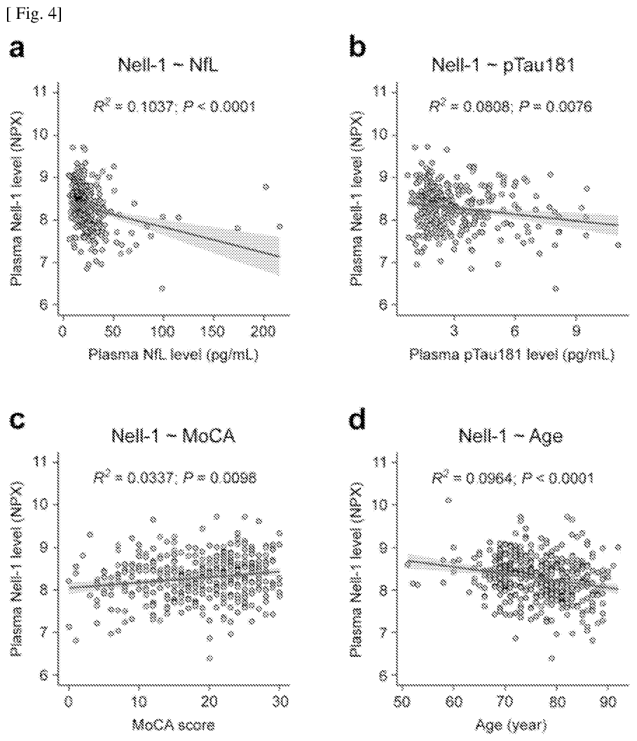

Nell-1 protein level was measured in plasma samples collected from the Hong Kong Chinese cohort. Plasma Nell-1 level is negatively associated with neurodegeneration indicated by plasma NfL level (β=−0.005, R²=0.1037, P<0.0001; FIG. 4a) and AD progression indicated by plasma pTau181 level (β=−0.043, R²=0.0808, P=0.0076; FIG. 4b). Moreover, plasma Nell-1 level is positively associated with cognitive performance indicated by MoCA score (β=0.012, R²=0.0337, P=0.0098; FIG. 4c) and negatively associated with aging (β=−0.014, R²=0.0964, P<0.0001; FIG. 4d). Taken together, Nell-1 protein level in blood can help monitor the progression of AD-related pathological changes in the brain.

### Example V: Attenuation of Neurodegeneration in the Brain Via Increasing Nell-1 Protein Level or Activity

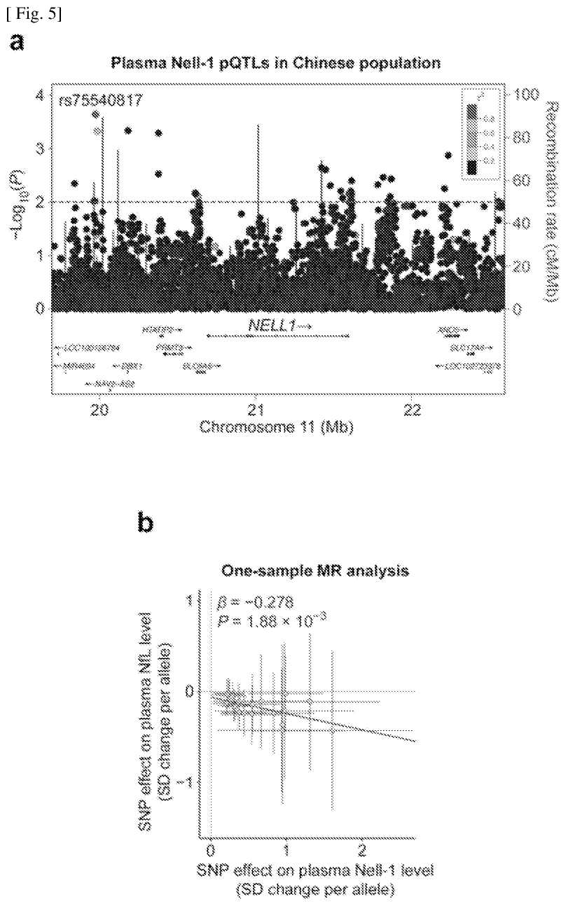

Nell-1 protein level was measured in plasma samples collected from the Hong Kong Chinese cohort. Whole genomes were also sequenced from DNA samples collected from the Hong Kong Chinese cohort. Genetic variants that modulate with plasma Nell-1 protein level were identified (P<0.01; FIG. 5a). Instrumental genetic variants were used to analyze the causal relationship between plasma Nell-1 level and AD-related endophenotypes via Mendelian randomization (MR) analysis. The MR analysis demonstrated that having elevated plasma Nell-1 level has causal effect on having reduced neurodegeneration indicated by lower plasma NfL level (β=−0.278, P=0.0019; FIG. 5b), demonstrating the protective effect of Nell-1 protein against neurodegeneration.

### Example VI: Improvement of Neuronal Functions in the Brain Via Increasing Nell-1 Protein Level or Activity

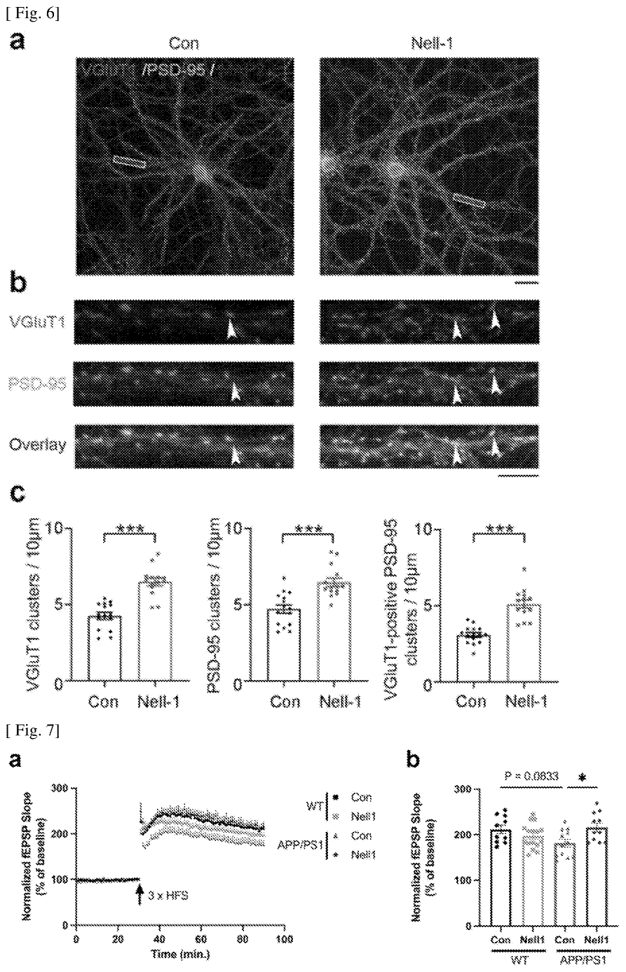

Nell-1 protein was administered to cultured primary rat hippocampal neurons. Nell-1 treatment led to a significant increase in the expression of presynaptic proteins and postsynaptic proteins in neuronal processes (P<0.001; FIGS. 6a-c). Moreover, Nell-1 treatment led to a significant increase in the number of excitatory synapses indicated by overlapping presynaptic and postsynaptic proteins (P<0.001; FIGS. 6a-c). Taken together, Nell-1 administration to neurons elicits beneficial effects on neuronal and synaptic functions.

### Example VII: Improvement of Synaptic Plasticity in the Brain Via Increasing Nell-1 Protein Level or Activity

Nell-1 protein was intracerebroventricularly administered for 7 days to the brains of 11-month-old wildtype (WT) or APP/PS1 mice (i.e. transgenic mouse models of AD). Nell-1 treatment rescued the impairment in hippocampal long-term potentiation (LTP) in APP/PS1 mice (P<0.05; FIG. 7a, b). LTP is a form of synaptic plasticity that involves the long lasting increase in synaptic plasticity and is associated with memory. Taken together, Nell-1 in vivo treatment leads to beneficial effects on synaptic plasticity.

### Example VIII: Improvement of Neurogenesis in the Brain Via Increasing Nell-1 Protein Level or Activity

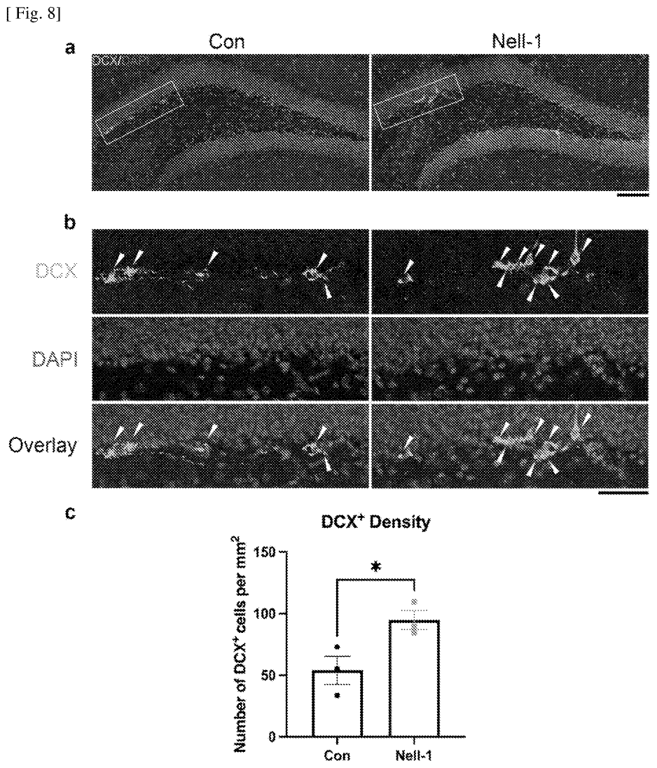

Nell-1 protein was intracerebroventricularly administered for 7 days to the brains of 11-month-old WT mice. Nell-1 treatment led to the increase in immature neuron density in the hippocampal dentate gyrus (P<0.05; FIG. 8a-c). The findings suggest that Nell-1 treatment exerts beneficial effects on neurogenesis.

All patents, patent applications, and other publications, including GenBank Accession Numbers and equivalents, cited in this application are incorporated by reference in the entirety for all purposes.

1. A method for diagnosing, monitoring, or assessing risk for a neurodegenerative disorder in a subject, comprising:
(1) comparing the subject's plasma or serum or whole blood level of Nell-1 protein with a standard control level of Nell-1 protein found in the plasma or serum or whole blood of an average healthy subject not suffering from or at increased risk for the neurodegenerative disorder; and
(2) detecting a decrease in the subject′ plasma or serum or whole blood level of Nell-1 protein from the standard control level, and determining the subject as suffering from or having an increased risk for the neurodegenerative disorder; or
(3) detecting no decrease in the subject' plasma or serum or whole blood level of Nell-1 protein from the standard control level, and determining the subject as not suffering from or having no increased risk for the neurodegenerative disorder.

2. The method of claim 1, further comprising, prior to step (1), measuring the plasma or serum or whole blood level of Nell-1 protein.

3. The method of claim 2, further comprising, prior to the measuring step, obtaining a plasma or serum or whole blood sample from the subject.

4. The method of claim 1, further comprising, after step (3), comparing the subject's plasma or serum or whole blood level of Nell-1 protein as measured at a later time with the subject's plasma or serum or whole blood level of Nell-1 protein in step (1), wherein a higher plasma or serum or whole blood level of Nell-1 protein as measured at the later time indicates improvement of the neurodegenerative disorder, and wherein a lower plasma or serum or whole blood level of Nell-1 protein as measured at the later time indicates deterioration of the neurodegenerative disorder.

5. The method of claim 4, wherein between step (3) and the later time the subject has been administered a therapeutic agent intended for treating the neurodegenerative disorder.

6. The method of any one of claims 1-5, wherein the neurodegenerative disorder is Alzheimer's Disease (AD).

7. The method of any one of claims 1-5, wherein the neurodegenerative disorder is Mild Cognitive Impairment (MCI) or Parkinson's Disease (PD).

8. A method for assessing severity or risk of a neurodegenerative disorder in two subjects, comprising:
(i) comparing the first subject's plasma or serum or whole blood level of Nell-1 protein with the second subject's plasma or serum or whole blood level of Nell-1 protein;
(ii) detecting the second subject's plasma or serum or whole blood level of Nell-1 protein lower than the first subject's plasma or serum or whole blood level of the Nell-1 protein; and
(iii) determining the second subject as having lower severity or lower risk of the neurodegenerative disorder than the first subject.

9. The method of claim 8, wherein the neurodegenerative disorder is Alzheimer's Disease (AD).

10. The method of claim 8, wherein the neurodegenerative disorder is Mild Cognitive Impairment (MCI) or Parkinson's Disease (PD).

11. The method of claim 8 or 9, further comprising, prior to step (i), measuring the plasma or serum or whole blood level of Nell-1 protein.

12. The method of claim 11, further comprising, prior to the measuring step, obtaining a plasma or serum or whole blood sample from the subject.

13. A kit for diagnosing a neurodegenerative disorder or assessing severity or risk of a neurodegenerative disorder in a subject, comprising a first reagent capable of determining the subject's plasma or serum or whole blood level of Nell-1 protein and optionally a second reagent capable of determining the subject's plasma or serum or whole blood level of phosphorylated-Tau-181 (pTau181) or neurofilament light polypeptide (NfL).

14. The kit of claim 12, comprising reagents capable of determining the subject's plasma or serum or whole blood level of pTau181 and NfL.

15. The kit of claim 12, further comprising a standard control reflecting the level of Nell-1 protein found in the plasma or serum or whole blood of an average healthy subject not suffering from and not having an increased risk for the neurodegenerative disorder.

16. The kit of any one of claims 12-15, wherein the neurodegenerative disorder is Alzheimer's Disease (AD), Mild Cognitive Impairment (MCI), or Parkinson's Disease (PD).

17. A method for treating or reducing risk for a neurodegenerative disorder, comprising administering to a subject in need thereof an effective amount of (1) a Nell-1 enhancing agent that enhances Nell-1 protein expression or activity; or (2) a Nell-1 receptor enhancing agent that enhances Nell-1 receptor expression or activity.

18. The method of claim 17, wherein the enhancing Nell-1 agent is Nell-1 protein or a nucleic acid encoding Nell-1 protein.

19. The method of claim 17, wherein the enhancing Nell-1 agent is a Nell-1 receptor, a nucleic acid encoding the Nell-1 receptor, or an agonist of the Nell-1 receptor.

20. The method of claim 17, wherein the administering comprises brain-targeting delivery of the Nell-1 enhancing agent or the Nell-1 receptor enhancing agent.

21. The method of claim 19 or 20, wherein the nucleic acid encoding Nell-1 protein or the Nell-1 receptor is formulated in a lipid nanoparticle composition for delivery.

22. The method of claim 19 or 20, wherein the nucleic acid encoding Nell-1 protein or the Nell-1 receptor is introduced by gene editing.

23. The method of any one of claims 17-22, further comprising, before and/or after the administering step, measuring the subject's plasma or serum or whole blood level of Nell-1 protein.

24. The method of claim 23, further comprising, prior to the measuring step or steps, obtaining a plasma or serum or whole blood sample from the subject.

25. The method of any one of claims 17-24, wherein the neurodegenerative disorder is Alzheimer's Disease (AD), Mild Cognitive Impairment (MCI), or Parkinson's Disease (PD).

26. A method for assessing efficacy of a therapeutic agent for treating a neurodegenerative disorder in a subject, comprising:
(1) comparing the subject's plasma or serum or whole blood level of Nell-1 protein before and after administration of the therapeutic agent to the subject;
(2) detecting an increase in the subject's plasma or serum or whole blood level of Nell-1 protein after administration of the therapeutic agent; and
(3) determining the therapeutic agent as effective for treating the neurodegenerative disorder.

27. The method of claim 26, wherein the neurodegenerative disorder is Alzheimer's Disease (AD), Mild Cognitive Impairment (MCI), or Parkinson's Disease (PD).

28. The method of claim 26 or 27, further comprising, prior to step (1), measuring the plasma or serum or whole blood level of Nell-1 protein before and after administration.

29. The method of claim 28, further comprising, prior to the measuring step, obtaining a plasma or serum or whole blood sample from the subject before and after administration.

30. The method of any one of claims 1-12 and 17-29, wherein the subject(s) is/are of Chinese descent.
